(19) United States
(12) Patent Application Publication
(10) Pub. No.: US 2021/0290173 A1
(43) Pub. Date: Sep. 23, 2021

(54) LATENT BIO-SIGNAL ESTIMATION USING BIO-SIGNAL DETECTORS

(71) Applicant: INTERNATIONAL BUSINESS MACHINES CORPORATION, ARMONK, NY (US)

(72) Inventors: Subhro Das, Cambridge, MA (US); Daby Mousse Sow, Croton on Hudson, NY (US)

(21) Appl. No.: 16/824,044

(22) Filed: Mar. 19, 2020

(51) Int. Cl. A61B 5/00; G16H 50/30

(52) U.S. Cl. CPC A61B 5/7264; A61B 5/0022; G16H 50/30; A61B 5/681; A61B 5/6898

(57) ABSTRACT

A latent bio-signal prediction can be obtained from at least one bio-signal from a consumer grade health monitoring device is described. A user's bio-signal readings from a health monitoring device can be analyzed by a model trained with the health records of a plurality of individuals. The model can be further personalized to user based on the individual traits of the user. The model can analyze the bio-signal and generate a latent bio-signal prediction. A latent bio-signal prediction can be transmitted to an electronic device for a user or health professional to monitor.

### BACKGROUND OF THE INVENTION

The present invention relates generally to measuring biological signals (bio-signals) and more specifically, to predicting bio-signals that are difficult or expensive to measure.

The past few years have seen a boom in popularity of wearable health monitoring devices. Many of these devices are capable of measuring many bio-signals, such as heart rate, blood oxygen saturation levels, and respiration rate, using an electrode and/or photoplethysmography (PPG). Unfortunately, wearable health monitoring devices are unreliable for continued use as biomarker measurement devices. Further, many bio-signals such as arterial oxygen saturation and intracranial pressure are latent, requiring invasive procedures or tests to acquire measurements.

### SUMMARY

Embodiments of the present disclosure include a method, computer program product, and system for predicting latent bio-signals. A processor can receive a first bio-signal of an individual user. The processor can analyze the first bio-signal. The processor can predict at least one latent bio-signal based on the analysis of the first bio-signal. The processor can send the latent bio-signal to an electronic device.

The above summary is not intended to describe each illustrated embodiment or every implementation of the present disclosure.

### BRIEF DESCRIPTION OF THE DRAWINGS

FIG. 1 is a functional block diagram generally depicting a latent bio-signal prediction environment, in accordance with an embodiment of the present invention.

FIG. 2 is a functional block diagram depicting a latent bio-signal prediction module, in accordance with an embodiment of the present invention.

FIG. 3 is a flowchart depicting operational steps of a method for predicting latent bio-signals using consumer grade health devices, in accordance with an embodiment of the present invention.

FIG. 4 is a block diagram of components of a prototype generation computer and a user prototype execution computer of an application prototype generation computing environment, in accordance with an embodiment of the present invention.

FIG. 5 is a block diagram depicting a cloud computing environment, in accordance with an embodiment of the present invention.

FIG. 6 is a block diagram depicting abstraction model layers, in accordance with an embodiment of the present invention.

### DETAILED DESCRIPTION

The embodiments depicted and described herein recognize the need for detecting certain bio-signals, which are normally only detectable by invasive procedures and/or expensive medical devices, hereinafter referred to as latent bio-signals.

In one embodiment of the invention, a smart watch can use a photoplethysmography (PPG) sensor or an electrode to read various bio-signals, such as, but not limited to, blood oxygen levels, heart rate, respiration rate, etc. of a user. The bio-signals can be used to create a model which can be further trained with bio-signals from other consumer grade health devices such as, but not limited to, a smart watch in an embodiment and the bio-signals from devices or tests which are more invasive in nature and difficult to obtain. Further, the model can be trained with health data of subjects producing latent bio-signals and bio-signals obtained by consumer health grade products. The model can further be configured specifically to the user wearing the smart watch producing the bio-signal. User personalization may allow the user to enter his or her physical data, medical data, personal data, and/or ancestry data. The data can be clustered to further increase the accuracy of a predicted latent bio-signal. The latent bio-signal can then be transmitted dynamically to the user's smart watch.

In another embodiment of the invention, a user can wear a consumer grade electrocardiogram (ECG) monitor, the ECG monitor can transmit bio-signal readings, such as heart rate, heart rhythm, derived heart rate variability, etc., to a smart watch or other suitable electronic device connected to a network or connected through a Bluetooth or ANT+ by Garmin. Further, using accelerometers in the ECG monitor the user's motion data, and activity data may be transmitted with the ECG sensor data. A noise filter may be incorporated to generate a more accurate reading by the ECG monitor based on filtering interference using a suitable Bayesian filter configured to reduce noise from interference such as, but not limited to, extraneous movement, electrical signals, etc. Bio-signals can be used in conjunction with health data from a variety of individuals. The health data may be from the structured data of an individual's health records and medical professional notes, including, but not limited to, ECG readings, pulse oximeter readings, cardiac stroke volume measurements, intracranial pressure measurements, etc. The ECG bio-signal can be analyzed to predict a latent bio-signal. The predicted latent signal is further refined by clustering the expected readings, based on the personal data provided by the user and assigning an expected outcome based on the readings. The predicted latent bio-signal can be transferred to the user's smart watch or electronic device dynamically or as a static output.

In describing embodiments in detail with reference to the figures, it should be noted that references in the specification to “an embodiment,” “other embodiments,” etc., indicate that the embodiment described may include a particular feature, structure, or characteristic, but every embodiment may not necessarily include the particular feature, structure, or characteristic. Moreover, such phrases are not necessarily referring to the same embodiment. Further, describing a particular feature, structure or characteristic in connection with an embodiment, one skilled in the art has the knowledge to affect such feature, structure or characteristic in connection with other embodiments whether or not explicitly described.

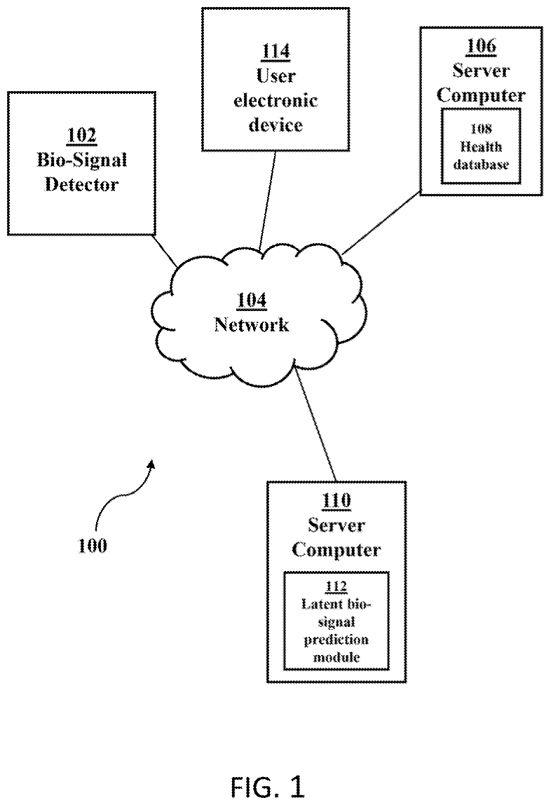

FIG. 1 is a functional block diagram illustrating, generally, an embodiment of a latent bio-signal prediction environment 100. The latent bio-signal prediction environment 100 comprises a bio-signal detector 102, a health data database 108 located on server computer 106, a latent bio-signal prediction module 112 operational on server computer 110, user electronic device 114 and a network 104 supporting communications between bio-signal detector 102, User Electronic Device 114, and server computers 108 and 110.

Bio-signal detector 102 can be a standalone bio-signal sensor device, for example, but not limited to a Whoop strap or a Rhythm+ by Scorche, or smart watch (e.g. Apple watch by Apple, Fenix 6 by Garmin, or Galaxy Watch by Samsung) or computing system including, but not limited to, smart watch and smart phone combinations capable of receiving, sending, and processing data associated with bio-signals. In another embodiment, bio-signal detector 102 can be an electrode, for example, but not limited to H10 by Polar or HRM-tri by Garmin, paired with a mobile computing device, smart watch, or smart phone capable of measuring bio-signals wherein the mobile computing device is capable of communicating with other computing devices (not shown) within the latent bio-signal prediction environment 100 via network 106.

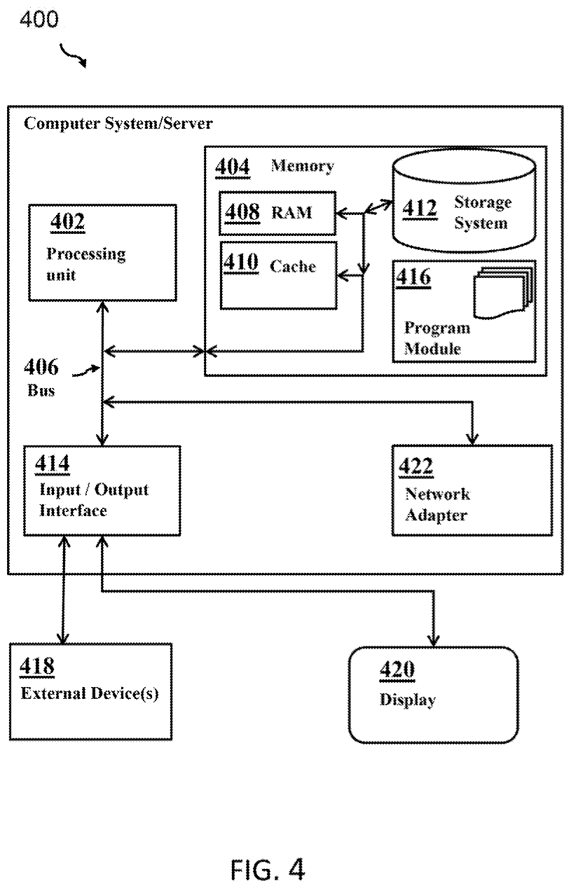

Bio-signal detector 102 can include internal and external hardware components, as depicted and described in further detail with respect to FIG. 4.

Bio-signal detector 102 can be a PPG sensor or electrode sensor. The bio-signals measurable by bio-signal detector 102 can include, but are not limited to, blood oxygen saturation, heart rate, blood pressure, cardiac output, respiration, arterial aging, endothelial function, microvascular blood flow, and other various autonomic functions.

Network 106 can be, for example, a local area network (LAN), a wide area network (WAN) such as the Internet, or a combination of the two, and can include wired, wireless, or fiber optic connections. In general, network 106 can be any combination of connections and protocols that will support communications between bio-signal detector 102 and server computers 106, 110.

Server computer 106, 110 can be a standalone computing device, management server, a web server, a mobile computing device, or any other electronic device or computing system capable of receiving, sending, and processing data. In other embodiments, server computer 106, 110 can represent a server computing system utilizing multiple computers as a server system. In another embodiment, server computer 106, 110 can be a laptop computer, a tablet computer, a netbook computer, a personal computer, a desktop computer, or any programmable electronic device capable of communicating with other computing devices (not shown) within latent bio-signal prediction environment 100 via network 104.

In another embodiment, server computer 106, 110 represents a computing system utilizing clustered computers and components (e.g., database server computers, application server computers, etc.) that act as a single pool of seamless resources when accessed within latent bio-signal prediction environment 100. Server computer 106, 110 can include internal and external hardware components, as depicted and described in further detail with respect to FIG. 4.

Health database 108 may be located on server computer 106. Health database 108 can be a database of individuals health records. The health records can be in electronic form. The health records can comprise data associated with, but not limited to, blood oxygen saturation, heart rate, blood pressure, cardiac output, respiration, arterial aging, endothelial function, microvascular blood flow, and other various autonomic functions. Additionally, the health records may include results and test data for more invasive and/or hazardous bio-signal tests, such as intracranial pressure, arterial blood gas, blood sugar, or similar tests. Further, the health records may include demographic information about the individuals, such as, but not limited to, age, ethnicity, height, weight, body mass index, body fat percentage, employment, and other similar information.

Latent bio-signal prediction module 112 may be operable on server computer 110, however, latent bio-signal prediction module 112 may be operational on bio-signal detector 102, user electronic device 114, or another suitable general computing device.

User electronic device 114 can be a smartphone, for example, but not limited to iPhone 11 by Apple, Galaxy 10 by Samsung, Pixel 4 by Google. User electronic device 114 may also be a smart watch for example, but not limited to Apple Watch by Apple, Galaxy watch by Samsung, Fenix 6 by Garmin. Additionally, user electronic device 114 may be a general computing device capable of communicating over network 104.

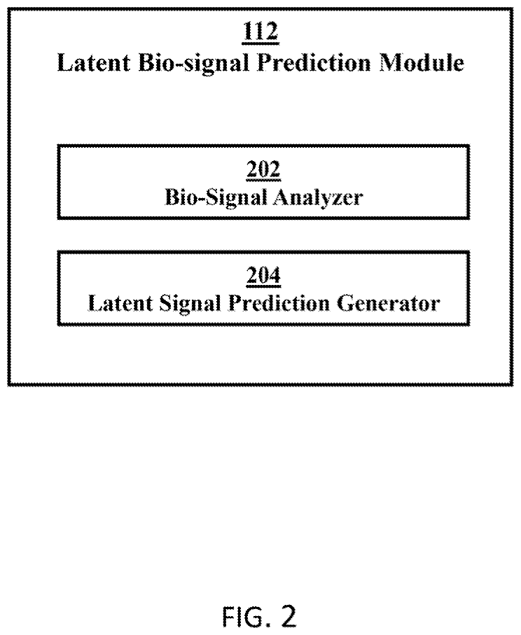

FIG. 2 is a functional block diagram depicting Latent bio-signal prediction module 112 comprising, bio-signal analyzer 202, and latent bio-signal prediction generator 204.

Bio-signal analyzer 202 receives and extracts the bio-signal data from bio-signal detector 102. Bio-signal analyzer 202 filter's out interference or noise from the bio-signal detector 102, such as light interference in a PPG sensor, signal interference or electrical interference in an electrode. This task can be accomplished using a Kalman filter, particle filter, or any other filter suited to remove interference from bio-signal detectors. The data output from the bio-signal analyzer 202 may be represented as y, where y is the dynamic bio-signal measured by the consumer grade medical device. Further, the bio-signal analyzer 202 may provide the capability to analyze other information from the bio-signal detector 102, such as, but not limited to, activity information or data readings from a multi-axis accelerometer located within or paired to bio-signal detector 102.

Latent signal prediction generator 204 is trained with the health data of a plurality of individuals. The health data can include but is not limited to survey data, demographic data, bio-signals from PPGs and electrodes and latent bio-signals. The bio-signals from PPGs can include, but are not limited to heart rate, cardiac cycle, respiration, peripheral oxygen saturation, and blood pressure. The bio-signals from electrodes can include, but are not limited to include cardiac cycle, heart rate, blood pressure. Further, the latent bio-signals used to train the model can include but are not limited to intracranial pressure, arterial oxidation saturation, and oxygen consumption.

Latent signal prediction generator 204 may be trained with the health data of the immediate user to allow for the personalization of the prediction generator. This involves the user inputting data for example, but not limited to, age, weight, biological gender, or allowing latent signal prediction generator 204 access to her medical records, provided user has given permission to access medical records as required by local, state, and federal laws. Data input by the user or gathered from the user's medical records would allow latent signal prediction generator 204 to cluster data using a clustering model, for example but not limited to K-means clustering, mean-shift, or clustering density-based spatial clustering of applications with noise.

Latent signal prediction generator 204 can predict latent bio-signals dynamically or on an interval basis. A model equation for the continuous state-space representation of physiological systems may be represented as follows: the latent bio-signal data may be represented by the following equation:

\dot{x}(t)=f(x(t),v(t))

where \dot{x}(t) represents the rate of change (with respect to time t) of a latent bio-signal, x(t) represents the latent dynamic physiological signals that are difficult to measure and v(t) represents other latent dynamic bio-signals that cannot be observed but influence the dynamics of x(t) for example but not limited to, cardiac stroke volume, arterial oxygen content, intracranial pressure, and nerve electromyography, f is the state/system function that maps x(t) and v(t) to the rate of change of the bio-signal x(t). Additionally, the observed bio-signals may be represented by the following equation:

y(t)=h(x(t),r(t))

where y(t) is the dynamic bio-signals observed by bio-signal detectors and r(t) are the observations of the noises of bio-signal detectors, h is the output function that maps x(t) and r(t) to the observed bio-signal y(t).

Model equations of latent signal prediction generator 204 to represent linear discrete-time representation for the dynamical-observation systems may be as follows:

Xₖ₊₁=Axₖ+Bvₖvₖ˜N(0,V)

and

yₖ=CxₖDrₖrₖ˜N(0,R)

where: xₖ is the latent bio-signal, k is the discrete time index, A is the system dynamics matrix, B is the system input matrix, and N signifies Gaussian distribution. In this representation, vₖ is the variability of bio-signals that cannot be measured, for example, but not limited to stress level or mood, and it follows Gaussian distribution with mean zero and system input covariance V. For the second equation, yₖ is the observed bio-signal, C is the observation matrix, D is the noise matrix, rₖ is the noise observed by the consumer grade health device for example, but not limited to light interference with the PPG, incorrect positioning of a device, or peak detection errors of a PPG or electrode, and, rₖ follows Gaussian distribution with mean zero and observation noise covariance R. Further, the output from probability generator may possess a machine learning capability by reinforcing accurate latent bio-signals in an iterative process in a deep learning model.

Latent signal prediction generator 204 can transmit the predicted latent bio-signal to the user electronic device 114 via the network 106. The predicted latent bio-signal may be transmitted by an email, text message or application located on user electronic device 114. Latent signal prediction generator 204 can have the capability to send a warning to user electronic device 114 if the latent bio-signal is falls in a predetermined dangerous range.

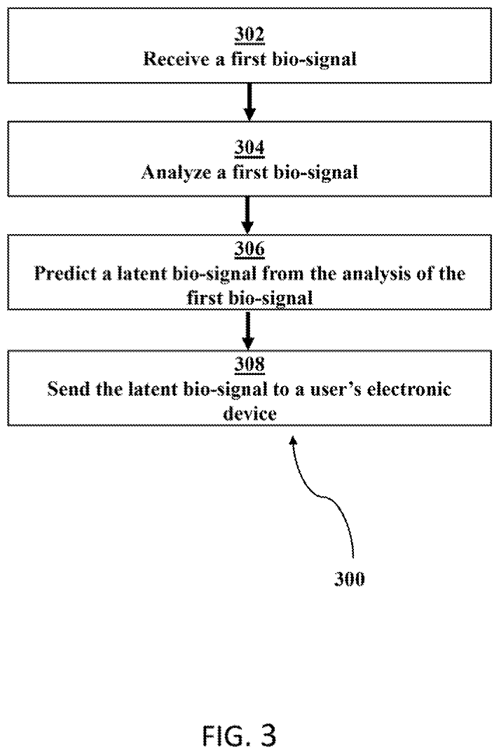

FIG. 3 is a flowchart of a method 300 depicting operational steps to predict a latent bio-signal. First, at step 302 receive a first bio-signal from bio-signal detector 102, at latent bio-signal prediction module 112. Next, at step 304 analyze the first bio-signal with bio-signal analyzer 202. Next, at step 306, predict a latent bio-signal from the analysis of the first bio-signal with latent bio-signal prediction generator 204, trained with health data 108. Next, at step 308, send the latent bio-signal to electronic device 114.

FIG. 4 shows, as an example, a computing system 400 suitable for executing program code related to the proposed method.

The computing system 400 is only one example of a suitable computer system and is not intended to suggest any limitation as to the scope of use or functionality of embodiments of the invention described herein, regardless, whether the computer system 400 is capable of being implemented and/or performing any of the functionality set forth hereinabove. In the computer system 400, there are components, which are operational with numerous other general purpose or special purpose computing system environments or configurations. Examples of well-known computing systems, environments, and/or configurations that may be suitable for use with computer system/server 400 include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like. Computer system/server 400 may be described in the general context of computer system-executable instructions, such as program modules, being executed by a computer system 400. Generally, program modules may include routines, programs, objects, components, logic, data structures, and so on that perform particular tasks or implement particular abstract data types. Computer system/server 400 may be practiced in distributed cloud computing environments where tasks are performed by remote processing devices that are linked through a communications network. In a distributed cloud computing environment, program modules may be located in both, local and remote computer system storage media, including memory storage devices.

As shown in the FIG. 4, computer system/server 400 is shown in the form of a general-purpose computing device. The components of computer system/server 400 may include, but are not limited to, one or more processors or processing units 402, a system memory 404, and a bus 406 that couple various system components including system memory 404 to the processor 402. Bus 406 represents one or more of any of several types of bus structures, including a memory bus or memory controller, a peripheral bus, an accelerated graphics port, and a processor or local bus using any of a variety of bus architectures. By way of example, and not limiting, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus. Computer system/server 400 typically includes a variety of computer system readable media. Such media may be any available media that is accessible by computer system/server 400, and it includes both, volatile and non-volatile media, removable and non-removable media.

The system memory 404 may include computer system readable media in the form of volatile memory, such as random-access memory (RAM) 408 and/or cache memory 410. Computer system/server 400 may further include other removable/non-removable, volatile/non-volatile computer system storage media. By way of example only, a storage system 412 may be provided for reading from and writing to a non-removable, non-volatile magnetic media (not shown and typically called a ‘hard drive’). Although not shown, a magnetic disk drive for reading from and writing to a removable, non-volatile magnetic disk (e.g., a ‘floppy disk’), and an optical disk drive for reading from or writing to a removable, non-volatile optical disk such as, but not limited to, a CD-ROM, DVD-ROM or other optical media may be provided. In such instances, each can be connected to bus 406 by one or more data media interfaces. As will be further depicted and described below, memory 104 may include at least one program product having a set (e.g., at least one) of program modules that are configured to carry out the functions of embodiments of the invention.

The program/utility, having a set (at least one) of program modules 416, may be stored in memory 404 by way of example, and not limiting, as well as an operating system, one or more application programs, other program modules, and program data. Each of the operating systems, one or more application programs, other program modules, and program data or some combination thereof, may include an implementation of a networking environment. Program modules 416 generally carry out the functions and/or methodologies of embodiments of the invention, as described herein.

The computer system/server 400 may also communicate with one or more external devices 418 such as a keyboard, a pointing device, a display 420, etc.; one or more devices that enable a user to interact with computer system/server 400; and/or any devices (e.g., network card, modem, etc.) that enable computer system/server 400 to communicate with one or more other computing devices. Such communication can occur via input/output (I/O) interfaces 414. Still yet, computer system/server 400 may communicate with one or more networks such as a local area network (LAN), a general wide area network (WAN), and/or a public network (e.g., the Internet) via network adapter 422. As depicted, network adapter 422 may communicate with the other components of the computer system/server 400 via bus 406. It should be understood that, although not shown, other hardware and/or software components could be used in conjunction with computer system/server 400. Examples, include, but are not limited to: microcode, device drivers, redundant processing units, external disk drive arrays, RAID systems, tape drives, and data archival storage systems, etc.

Embodiments of the invention may be implemented together with virtually any type of computer, regardless of the platform being suitable for storing and/or executing program code. FIG. 4 shows, as an example, a computing system 400 suitable for executing program code related to the proposed method.

The computing system 400 is only one example of a suitable computer system and is not intended to suggest any limitation as to the scope of use or functionality of embodiments of the invention described herein, regardless, whether the computer system 400 is capable of being implemented and/or performing any of the functionality set forth hereinabove. In the computer system 400, there are components, which are operational with numerous other general purpose or special purpose computing system environments or configurations. Examples of well-known computing systems, environments, and/or configurations that may be suitable for use with computer system/server 400 include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like. Computer system/server 400 may be described in the general context of computer system-executable instructions, such as program modules, being executed by a computer system 400. Generally, program modules may include routines, programs, objects, components, logic, data structures, and so on that perform particular tasks or implement particular abstract data types. Computer system/server 400 may be practiced in distributed cloud computing environments where tasks are performed by remote processing devices that are linked through a communications network. In a distributed cloud computing environment, program modules may be located in both, local and remote computer system storage media, including memory storage devices.

The descriptions of the various embodiments of the present invention have been presented for purposes of illustration but are not intended to be exhaustive or limited to the embodiments disclosed. Many modifications and variations will be apparent to those of ordinary skills in the art without departing from the scope and spirit of the described embodiments. The terminology used herein was chosen to best explain the principles of the embodiments, the practical application or technical improvement over technologies found in the marketplace, or to enable others of ordinary skills in the art to understand the embodiments disclosed herein.

The present invention may be embodied as a system, a method, and/or a computer program product. The computer program product may include a computer readable storage medium (or media) having computer readable program instructions thereon for causing a processor to carry out aspects of embodiments of the present invention.

The medium may be an electronic, magnetic, optical, electromagnetic, infrared or a semi-conductor system for a propagation medium. Examples of a computer-readable medium may include a semi-conductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W), DVD and Blu-Ray-Disk.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disk read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object-oriented programming language such as Smalltalk, C++ or the like, and conventional procedural programming languages, such as the “C” programming language or similar programming languages. The computer readable program instructions may execute entirely on the user's computer, partly on the user's computer as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatuses, or another device to cause a series of operational steps to be performed on the computer, other programmable apparatus or other device to produce a computer implemented process, such that the instructions which execute on the computer, other programmable apparatuses, or another device implement the functions/acts specified in the flowchart and/or block diagram block or blocks.

The flowcharts and/or block diagrams in the figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or act or carry out combinations of special purpose hardware and computer instructions.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

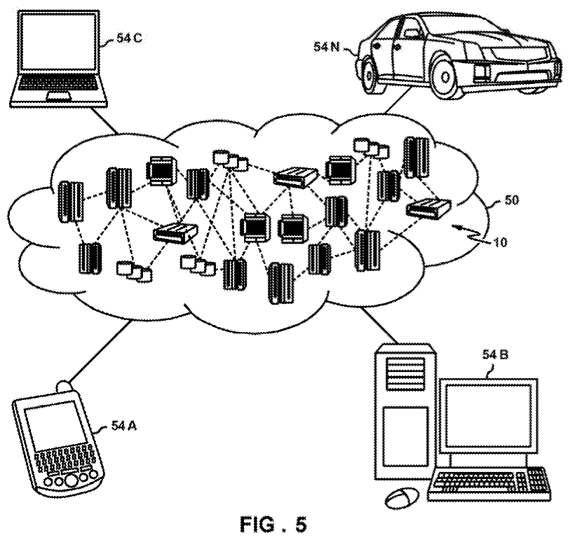

Referring now to FIG. 5, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 4 are intended to be illustrative only and that computing nodes 10 and cloud computing environment 50 can communicate with any type of computerized device over any type of network and/or network addressable connection (e.g., using a web browser).

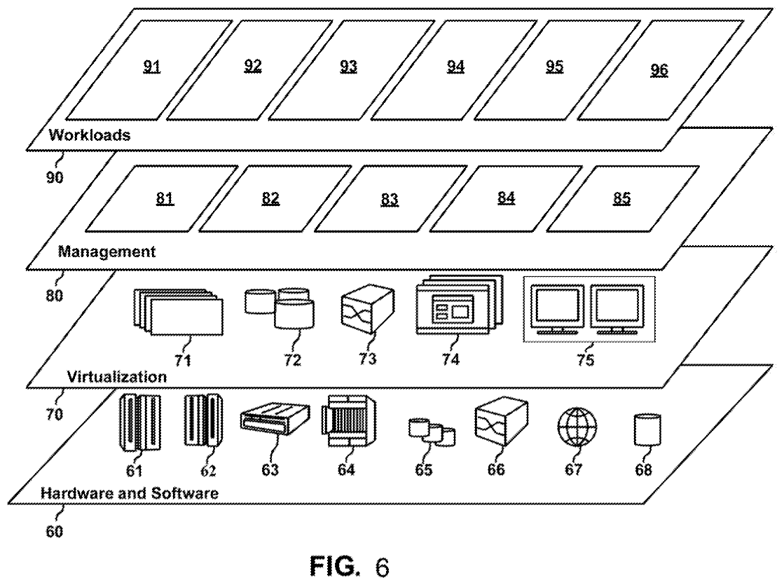

Referring now to FIG. 6, a set of functional abstraction layers provided by cloud computing environment 50 (FIG. 4) is shown. It should be understood in advance that the components, layers, and functions shown in FIG. 6 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and latent bio-signal prediction 96

The terminology used herein is for the purpose of describing particular embodiments only and is not intended to limit the invention. As used herein, the singular forms “a”, “an” and “the” are intended to include the plural forms as well, unless the context clearly indicates otherwise. It will further be understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or steps plus function elements in the claims below are intended to include any structure, material, or act for performing the function in combination with other claimed elements, as specifically claimed. The description of the present invention has been presented for purposes of illustration and description but is not intended to be exhaustive or limited to the invention in the form disclosed. Many modifications and variations will be apparent to those of ordinary skills in the art without departing from the scope and spirit of the invention. The embodiments are chosen and described in order to best explain the principles of the invention and the practical application, and to enable others of ordinary skills in the art to understand the invention for various embodiments with various modifications, as are suited to the particular use contemplated.

What is claimed is:

1. A computer implemented method for predicting latent bio-signals, the computer implemented method comprising:
receiving, by one or more processors, a first bio-signal of an individual user;
analyzing, by the one or more processors, the first bio-signal;
predicting, by one or more processors, at least one latent bio-signal based on the analysis of the first bio-signal; and
sending, by one or more processors, the latent bio-signal to an electronic device.

2. The computer-implemented method of claim 1, wherein the prediction of the first-bio signal is based on a personalized probabilistic clustering model.

3. The computer-implemented method of claim 2, further comprising:
receiving, by the one or more processors, the individual's health data.

4. The computer-implemented method of claim 2, further comprising:
sending the first bio-signal and latent bio-signal to a centralized database for continuous training of the personalized probabilistic clustering model.

5. The computer-implemented method of claim 2, wherein the personalized probabilistic clustering model is trained with historical data.

6. The computer-implemented method of claim 1, wherein the electronic device is a smart phone or smart watch.

7. The computer-implemented method of claim 1 wherein the first bio-signal of a user is at least one of the following, heartrate, cardiac cycle, respiration rate, and peripheral oxygen saturation.

8. A computer system for predicting a latent bio-signal from a bio-signal, the computer system comprising:
one or more computer processors;
one or more non-transitory computer readable storage media;
program instructions stored on the at least one or more non-transitory computer readable storage media for execution by at least one of the one or more computer processors, the program instructions comprising:
program instructions to receive a first bio-signal of an individual;
program instructions to analyze the first bio-signal;
program instructions to predict a latent bio-signal based on the analysis of the first bio-signal; and
program instructions to send the latent bio-signal to an electronic device.

9. The computer system of claim 8, wherein the prediction of the analysis of the first bio-signal is based on a personalized probabilistic clustering model.

10. The computer system of claim 9, further comprising:
program instructions to receive the individual's health data.

11. The computer system of claim 9 further comprising:
program instructions to send the first bio-signal and predicted latent bio-signal to a centralized database for continuous training of the personalized probabilistic clustering model.

12. The computer system of claim 9 wherein, the probabilistic clustering model is trained with historical data.

13. The computer system of claim 8 wherein, the electronic device of the user is a smart phone or smart watch.

14. The computer system of claim 8 wherein, the first bio-signal of a user is at least one of the following, heart rate, cardiac cycle, respiration rate, and peripheral oxygen saturation.

15. A computer program product for predicting a latent bio-signal from a bio-signal measured with a consumer grade health device, the computer program product comprising one or more computer readable storage media and program instructions sorted on the one or more computer readable storage media, the program instructions including instructions to:
receive a first bio-signal of an individual;
analyze the first bio-signal;
predict a latent bio-signal based on the analysis of the first bio-signal; and
send the latent bio-signal to an electronic device.

16. The computer program product of claim 15, wherein the prediction of the analysis of the first bio-signal is based on a personalized probabilistic clustering model.

17. The computer program product of claim 16, further comprising instructions to receive the individual's health data.

18. The computer program product of claim 17 further comprising instructions to send the first bio-signal and predicted latent bio-signal to a centralized database for continuous training of the personalized probabilistic clustering model.

19. The computer program product of claim 16 wherein, the probabilistic clustering model is trained with historical data.

20. The computer program product of claim 15 wherein, the electronic device of the user is a smart phone or smart watch.
